(19) United States
(12) Patent Application Publication
(10) Pub. No.: US 2004/0241234 A1
(43) Pub. Date: Dec. 2, 2004

(54) Controlled release press-coated formulations of water-soluble active agents

(75) Inventor: Zalman Vilkov, Dingman's Ferry, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: ALPHARMA, INC., Fort Lee, NJ

(21) Appl. No.: 10/452,754

(22) Filed: Jun. 2, 2003

(51) Int. Cl. A61K 9/24; A61K 9/16; A61K 9/50

(52) U.S. Cl. 424/471

(57) ABSTRACT

A press-coat formulation comprising a core composition comprising a water-soluble active agent and a waxy material and a coating composition comprising the active agent and a polymer, wherein the coating composition is press-coated onto the core.

### FIELD OF THE INVENTION

The invention relates to controlled release press-coated formulations of water-soluble active agents.

### BACKGROUND OF THE INVENTION

Controlled delivery of active agents is often desirable, especially with respect to oral delivery of pharmaceutically active agents to patients. Controlled delivery of pharmaceutically active agents, for example, helps avoid the overdosing or under-dosing that can result from uncontrolled release. Attempts have been made to achieve controlled delivery of various active agents. However, each of these attempts have suffered from several drawbacks.

Oral osmotic delivery systems can require substantial amounts of organic solvent to prepare the coating that provides controlled release of the active agent. In addition, a hole must be provided in each tablet to allow the active agent to be dissolved and released from the tablet in a controlled manner. Further, the spent tablet undesirably retains its size after delivery of the active agent which the patient must pass.

Erodible matrices can provide suitable controlled release of water-insoluble active agents. However, controlled release of water soluble active agents is difficult because, among other things, the active agent embedded in the matrix is likely to dissolve at a rate faster than the rate the matrix erodes.

Active coated inert spheres can provide suitable controlled release of an active agent in lower dosages, but are costly. In addition, for larger doses, coated inert spheres are undesirable because, among other things, a significant portion of the volume of the core is taken up by the non-active sphere core particle.

Standard control release coatings on active agent core formulations also have problems, especially with highly water-soluble drugs. Aqueous coating solutions can result in redistribution and early degradation of the active agent. Further, organic solvents in the coating process are costly and pose safety and environmental concerns.

Press-coated tablets are also problematic because, for example, the core must be placed precisely in the center of the die. A core that is off slightly to one side or the other could result in the uncontrolled delivery of the active agent. For larger doses, press-coated tablets are undesirable because the large off-center core results in the core being extremely close to the outer boundary of the press-coat, or even not coated at all by the press-coat.

The invention overcomes these and other problems by providing a press-coat formulation of water-soluble active agents in a controlled release core which is press-coated with a controlled release coating formulation also comprising the water-soluble active agents therein.

### BRIEF SUMMARY OF THE INVENTION

The invention provides a press-coat composition comprising (a) a core composition comprising a water-soluble active agent and a waxy material and (b) a coating composition comprising the water-soluble active agent and a hydrophilic polymer, wherein the coating composition is press-coated onto the core.

In certain preferred embodiments of the invention is provided a press-coat composition comprising (a) a core composition comprising an active agent and carnauba wax and (b) a coating composition comprising the active agent and hydroxypropylmethyl cellulose (HPMC), wherein the active agent is selected from the group consisting of a pharmaceutically acceptable salt of methylphenidate, fluoxetine, oxybutynin, and mixtures thereof, and wherein the coating composition is press-coated onto the core.

In certain other preferred embodiments is provided a press-coat composition comprising (a) a core composition comprising a water-soluble active agent and a waxy material, (b) a coating composition comprising the water-soluble active agent and a hydrophilic polymer, wherein the coating composition is press-coated onto the core, and (c) an additional coating composition comprising the water-soluble active agent.

In a preferred embodiment of the invention is provided a press-coat composition comprising (a) a core composition comprising a pharmaceutically acceptable salt of methylphenidate and a waxy material and (b) a coating composition comprising the pharmaceutically acceptable salt of methylphenidate and a hydrophilic polymer, wherein the coating composition is press-coated onto the core.

In another preferred embodiment of the invention is provided a press-coat composition comprising (a) a core composition comprising methylphenidate HCl and carnauba wax, (b) a coating composition comprising methylphenidate HCl and hydroxypropylmethyl cellulose (HPMC), wherein the coating composition is press-coated onto the core.

A still further embodiment of the invention provides a press-coat composition comprising (a) a core composition comprising methylphenidate HCl and carnauba wax, (b) a coating composition comprising methylphenidate HCl and hydroxypropylmethyl cellulose (HPMC), wherein the coating composition is press-coated onto the core, and (c) an additional coating composition comprising methylphenidate HCl.

A method is provided for preparing a press-coat composition, the method comprising (a) providing a core composition comprising a water-soluble active agent and a waxy material, (b) providing a coating composition comprising the water-soluble active agent and a hydrophilic polymer, and (c) press-coating the coating composition onto the core composition to provide the press-coat composition.

### BRIEF DESCRIPTION OF THE DRAWINGS

FIGS. 1a-1c show release rates of certain preferred embodiments of the invention comprising certain pharmaceutically acceptable active agents.

FIG. 2 shows release rates of certain preferred embodiments of the invention comprising various amounts of controlled release agents in the press-coat formulation.

FIG. 3 shows substantially zero-, first- and second-order release rates of certain preferred embodiments of the invention comprising various amounts of the active agent in the core and coating formulations.

FIG. 4 shows release rates of certain preferred embodiments of the invention tested at various rotation speeds.

FIG. 5 shows a substantially zero-order release rate of a certain preferred embodiment of the invention.

FIG. 6 shows a substantially second-order release rate of a certain preferred embodiment of the invention.

FIG. 7 shows a substantially zero-order release rate of a certain preferred embodiment of the invention.

### DESCRIPTION OF THE PREFERRED EMBODIMENT OF THE INVENTION

In a preferred embodiment of the invention is provided a dosage form of a water-soluble active agent, the dosage form comprising a core and a coating formulation press-coated on the core, wherein the core comprises a waxy material and the water-soluble active agent and wherein the coating composition comprises a hydrophilic polymer and the water-soluble active agent.

By “water-soluble” active agent is meant to include active agents that are at least slightly water-soluble (for example, about 1 to about 10 mg/ml at 25° C.). In a preferred embodiment of the invention, the active agent is moderately water-soluble (for example, less than about 100 mg/ml at 25° C.). In another preferred embodiment of the invention, the active agent is highly water-soluble (for example, greater than about 100 mg/ml at 25° C.). Preferably, the active agent is highly water soluble.

The active agents of the invention include any active agent for which controlled release is desirable. In a preferred embodiment of the invention, the active agent is a pharmaceutically acceptable active agent and includes pharmaceutical and veterinary active agents (often referred to as drugs). In other embodiments, the active agent includes agrichemical agents (such as fertilizers, herbicides, pesticides and fungicides), active agent used in the exterminating industry (such as toxins and poisons), and active agents used in industrial manufacturing (such as catalysts or catalytic quenchers).

Exemplary active agents for use in the pharmaceutical and veterinary applications of the invention include analgesics, anesthetics, anticonvulsants, antidiabetic agents, antihistamines, anti-infectives, antineoplastics, antiparkinsonian agents, antirheumatic agents, appetite stimulants, appetite suppressants, blood modifiers, bone metabolism modifiers, cardiovascular agents, central nervous system depressants, central nervous system stimulants, decongestants, dopamine receptor agonists, electrolytes, gastrointestinal agents, immunomodulators, muscle relaxants, narcotics, parasympathomimetics, sympathomimetics, sedatives, and hypnotics.

In certain preferred embodiments of the invention, the active agent includes methylphenidate, fluoxetine, or oxybutynin. More preferably, the active agent includes pharmaceutically acceptable salts of methylphenidate (including the B-threo and L-threo forms and mixtures thereof), fluoxetine or oxybutynin. Most preferably, the active agent is selected from methylphenidate hydrochloride, fluoxetine hydrochloride, and oxybutynin chloride. Other pharmaceutically acceptable active agents having solubility in water similar to these preferred compounds are also contemplated to be within the scope of the invention.

The term “active agent” is meant to include solvates (including hydrates) of the free compound or salt, crystalline and non-crystalline forms, as well as various polymorphs. For example, the active agent “methylphenidate” can include all optical isomers of the compound and all pharmaceutically acceptable salts thereof either alone or in combination (the combined threo isomers can be indicated as “threo” and the combined erythro isomers as “erythro”). In a preferred embodiment of the invention, the methylphenidate is a dl-threo methylphenidate hydrochloride. More preferably, the methylphenidate is a d-threo methylphenidate hydrochloride.

In a preferred embodiment of the invention, a core comprising an active agent and a waxy controlled release agent is coated with a press-coat formulation also comprising the active agent and a hydrophilic polymer controlled release agent.

The invention provides drug formulations having substantially zero order, first order, and second order release rate profiles by adjusting the amount of active agent in the core and the press-coat formulation. The ratio of the active agent in the core (Core_{AA}) to active agent in the press-coat (Coat_{AA}) preferably ranges from about 1:99 to about 99:1, more preferably from about 95:5 to about 5:99, most preferably from about 9:1 to about 1:9. For the highly soluble active agents, including methylphenidate hydrochloride, fluoxetine hydrochloride, and oxybutynin chloride, a Core_{AA}:Coat_{AA} of about 3:4 to about 5:3 is preferred to provide a substantially zero order release rate, a Core_{AA}:Coat_{AA} of less than about 3:4 is preferred to provide a substantially first order release rate, and a Core_{AA}:Coat_{AA} of greater than about 5:3 is preferred to provide a substantially second order release rate.

The waxy material is preferably selected from hydrophobic waxy materials to provide controlled release of the active agent. In pharmaceutical and/or veterinary products, for example, such waxy materials are preferably selected from materials such as carnauba wax, tribehenin, fatty alcohols (particularly those having 12-24 carbon atoms, such as lauryl alcohol, myristyl alcohol, stearyl alcohol, palmityl alcohol, etc such as stearyl alcohol), fatty acids (particularly those having 12-24 carbon atoms, such as lauric acid, myristic acid, stearic acid, palmitic acid, etc), polyethylenes, castor wax, C₁₆₋₃₀ fatty acid triglycerides, beeswax, and mixtures thereof. Preferably, the core comprises carnauba wax.

In a preferred embodiment of the invention, the core further includes a filler, such as a water insoluble filler, water soluble filler, and mixtures thereof. Preferably, the filler is a water-insoluble filler, such as calcium salt or talc. In a preferred embodiment, the calcium salt is a calcium phosphate, more preferably a dicalcium phosphate. Exemplary water-soluble fillers include water soluble sugars and sugar alcohols, preferably lactose, glucose, fructose, sucrose, mannose, galactose, the corresponding sugar alcohols and other sugar alcohols, such as mannitol, sorbitol, and xylitol.

Other excipients can also be present in the core formulation, including lubricants (such as talc and magnesium stearate), glidants (such as fumed or colloidal silica), pH modifiers (such as acids, bases and buffer systems), and pharmaceutically useful processing aids.

In a preferred embodiment of the invention, the core components (active agent, wax and optional excipients) are blended together and compressed into suitable cores. The blending can take place in any order of addition. Preferably, the cores are blended by starting with the smallest volume component and then successively adding the larger volume components. Another preferred process is to melt the wax and to blend the active agent and optional excipients into the melted wax. Alternatively, the active agent, wax and optional excipients can be blended together and then subjected to a temperature at which the wax will melt. Once cooled, the solidified mass can be milled into granules for compaction into cores.

In a preferred embodiment of the invention, the hydrophilic polymer in the press-coat composition provides for the controlled release of the active agent. Preferably, the hydrophilic polymer for providing controlled release is a film forming polymer, such as a hydrophilic cellulose polymer. Preferably, the hydrophilic cellulose polymer is a hydroxyalkyl cellulose polymer, such as hydroxyethylcellulose (HEC), hydroxypropyl cellulose (HPC), hydroxypropylmethylcellulose (HPMC), hydroxypropylethylcellulose (HPEC), hydroxypropylpropylcellulose (HPPC), and hydroxypropylbutylcellulose (HPBC). Most preferably, the controlled release agent is HPMC.

The press-coat composition can optionally include fillers, such as water soluble fillers, a water-insoluble fillers, and mixtures thereof. Preferably, the filler is water-soluble. Exemplary water soluble fillers include a water-soluble sugar, lactose, glucose, fructose, sucrose, galactose, mannose, the corresponding sugar alcohols and other sugar alcohols, such as mannitol, sorbitol, and xylitol. Most preferably, the water soluble filler is lactose or granulated mannitol. The water-insoluble fillers can include, for example, calcium salt or talc. A preferred insoluble calcium salt is a calcium phosphate, more preferably dicalcium phosphate. The filler in the press-coat composition can be the same or different as the filler in the core composition, if any. For example, the core composition can include a water soluble filler while the press coat composition can include a water-insoluble filler. In a preferred embodiment, the filler material is the same in both the core and the press-coat compositions.

The press-coat composition can optionally include other excipients, such as lubricants, processing aids, pH buffers, glidants, and the like, which can be the same or different as those in the core composition, if any.

In a preferred embodiment of the invention, the core composition is press-coated with the press-coat composition to form a tablet. The tablet can be further coated with optional additional coatings. The additional coatings can be pH-dependent or pH-independent, aesthetic or functional, and can include the active agent in immediate or controlled release. Such additional coatings preferably include film forming materials. In a preferred embodiment of the invention, the additional coating is an aesthetic coating. In another preferred embodiment, the additional coating includes an active agent, either the same or different as the active agent contained in the core and press-coat. Preferably, the active agent in the additional coating is an active agent that is desirably administered along with the active agent in the core and press-coat formulations. In other preferred embodiments, the additional coating includes an immediate release loading dose of the same active agent that is found in the core and press-coat formulations.

In certain preferred embodiments of the invention, 18 mg, 36 mg, and 54 mg methylphenidate hydrochloride tablets are prepared having one or more of a Tₘₐₓ of from about 3.5 to about 12 hours, preferably about 4.5 to about 10 hours, even more preferably about 6 to about 8 hours, and most preferably about 6.5 to about 7 hours; a Cₘₐₓ of from about 2.7 to about 4.9 ng/ml, preferably about 3.2 to about 4.2 ng/ml, more preferably about 3.4 to about 3.9 ng/ml, most preferably about 3.6 to about 3.8 ng/ml (for an 18 mg methylphenidate hydrochloride formulation); about 5 to about 10 ng/ml, preferably about 5.5 ng/ml to about 8 ng/ml, more preferably about 5.5 ng/ml to about 7.5 ng/ml, most preferably about 6 to about 6.5 ng/ml (for a 36 mg methylphenidate hydrochloride formulation); and about 8 to about 15 ng/ml, preferably about 8.5 to about 13 ng/ml, more preferably about 9 to about 12 ng/ml (for a 36 mg methylphenidate hydrochloride formulation); an AUC of from about 35 to about 45 (h)(ng/ml), preferably about 27.5 to about 43 (h)(ng/ml), more preferably about 41.6 to about 42 (h)(ng/ml) (for an 18 mg methylphenidate hydrochloride formulation), about 66.7 to about 70.5 (h)(ng/ml), more preferably 67.2 to about 70 (h)(ng/ml), most preferably about 67.6 (h)(ng/ml) (for a 36 mg methylphenidate hydrochloride formulation); and a four hour post administration blood level of about 1.75 to about 3.75 ng/ml, preferably about 1.9 to about 2.5 ng/ml, more preferably about 2 to about 2.25 ng/ml (for an 18 mg methylphenidate hydrochloride formulation) and about 5.5 to about 10 ng/ml, preferably about 6 to about 9 ng/ml (for a 54 mg methylphenidate hydrochloride formulation).

In a preferred embodiment of the invention, an oxybutynin chloride tablet is provided, wherein the release rate of the active agent is from about 0.05 mg/hr to about 0.85 mg/hr over a substantial portion of a 24 hour period, in a substantially zero-order release profile. Plasma concentrations can be maintained at levels over a substantial portion of the 24 hour once-daily administration period from about 4.2 ng/ml to about 6.7 ng/ml, and the ratio of oxybutynin to its desethyl metabolite can be maintained at a ratio of at least about 0.18:1. Additional information on oxybutynin formulations (non-press-coat) and oxybutynin responsive conditions can be found in U.S. Pat. No. 6,124,355; U.S. Pat. No. 5,840,754; U.S. Pat. No. 5,912,268; U.S. Pat. No. 6,262,115; and U.S. Pat. No. 5,674,895, and is incorporated herein by reference.

In a preferred embodiment of the invention is provided a substantially zero-order release dosage form. Preferably, the substantially zero-order release dosage form releases about 20 to about 50%, more preferably about 25 to about 50%, of the active agent released within about 4 hours and at least about 70%, preferably at least about 80%, of the active agent released within about 12 hours. In certain preferred embodiments of the invention comprising methylphenidate, the dosage form preferably releases about 25 to about 50% of the active agent within about 4 hours and at least about 80% within about 12 hours. In certain other preferred embodiments of the invention comprising fluoxetine, the dosage form preferably releases about 25 to about 50% of the active agent within about 4 hours and at least about 80% within about 12 hours. In those preferred embodiments of the invention comprising oxybutynin, the dosage form preferably releases about 20 to about 40% of the active agent within about 4 hours and at least about 70% within about 12 hours.

In another preferred embodiment of the invention is provided a substantially first-order release dosage form. Preferably, the substantially first-order release dosage form releases about 40 to about 70%, more preferably about 50 to about 70%, of the active agent within about 4 hours, and at least about 85%, preferably at least about 90%, within about 12 hours.

In still another preferred embodiment of the invention is provided a substantially second-order release dosage form. Preferably, the substantially second-order release dosage form releases about 5 to about 30%, more preferably about 15 to about 30%, of the active agent within about 4 hours, and at least about 75%, preferably at least about 90%, within about 18 hours. In certain embodiments of the invention comprising a methylphenidate as an active agent, a substantially second-order release rate can be obtained and is preferably from about 5 to about 30%, more preferably 15 to about 25% of the active agent released within about 4 hours, and at least about 75%, preferably at least about 85%, more preferably at least about 90%, of the active agent released within about 12 hours. In certain other preferred embodiments of the invention comprising a fluoxetine as an active agent, a substantially second-order release rate can be obtained and is preferably from about 5 to about 30%, more preferably about 15 to about 25%, of the active agent released within about 4 hours, and preferably at least about 75%, more preferably at least about 85%, most preferably at least about 90%, of the active agent released within about 12 hours. In still other preferred embodiments of the invention comprising oxybutynin, the dosage form releases about 5 to about 30%, preferably about 15 to about 25%, of the active agent within about 4 hours and at least about 75%, preferably at least about 85%, more preferably at least about 90%, of the active agent within about 18 hours.

In a preferred embodiment of the invention, a methylphenidate hydrochloride formulation is provided having 10 mg in an immediate release coating composition and 26 mg between the core composition and the press-coat composition. In this embodiment, the 0-4 hour cumulative release of active agent in 0.1 N hydrochloric acid is preferably at least about 25% to about 50%, more preferably about 35 to about 45%, of the loaded dose, and the 0-12 hour cumulative release of the active agent in 0.1 N hydrochloric acid is preferably at least about 80%, more preferably at least about 85%, of the dosage form dose. In another preferred embodiment of the invention, a 54 mg methylphenidate hydrochloride formulation is provided having a 3:2:1 (core:press coat:immediate release coat) ratio, e.g., a core comprising 27 mg of methylphenidate hydrochloride, a press-coat formulation comprising 18 mg of methylphenidate hydrochloride, and an immediate release loading dose comprising 9 mg of methylphenidate hydrochloride.

The following examples further illustrate the invention but, of course, should not be construed in any way limiting its scope.

### EXAMPLES

### Example 1

Press-coated tablets were prepared from cores of the following formulation:

| Component | mg per tablet |
|---|---|
| Methylphenidate HCl | 16.0 |
| Dicalcium Phosphate | 39.4 |
| Carnauba Wax | 24.0 |
| Cab-O-Sil (silica) | 0.2 |
| Magnesium Stearate | 0.4 |
| Total Core | 80.0 |

The components were blended together and compressed to form a core composition. The core composition was then press-coated with the composition below:

| Component | mg per tablet |
|---|---|
| Methylphenidate HCl | 10.0 |
| Lactose Monohydrate | 210.0 |
| METHOCEL K4M | 96.0 |
| Cab-O-Sil (silica) | 1.0 |
| Magnesium Stearate | 3.0 |
| Total Press-coat | 320.0 |

The press-coat composition components were blended together and about one half of the blend (about 160 mg) was placed in a die and lightly pressed. A core composition, as prepared above, was placed at approximately the center of the die and covered with the other half of the press-coat composition and pressed in the die to form a tablet. Some tablets were then given a further immediate release “loading dose” of an additional 10 mg of methylphenidate hydrochloride per tablet.

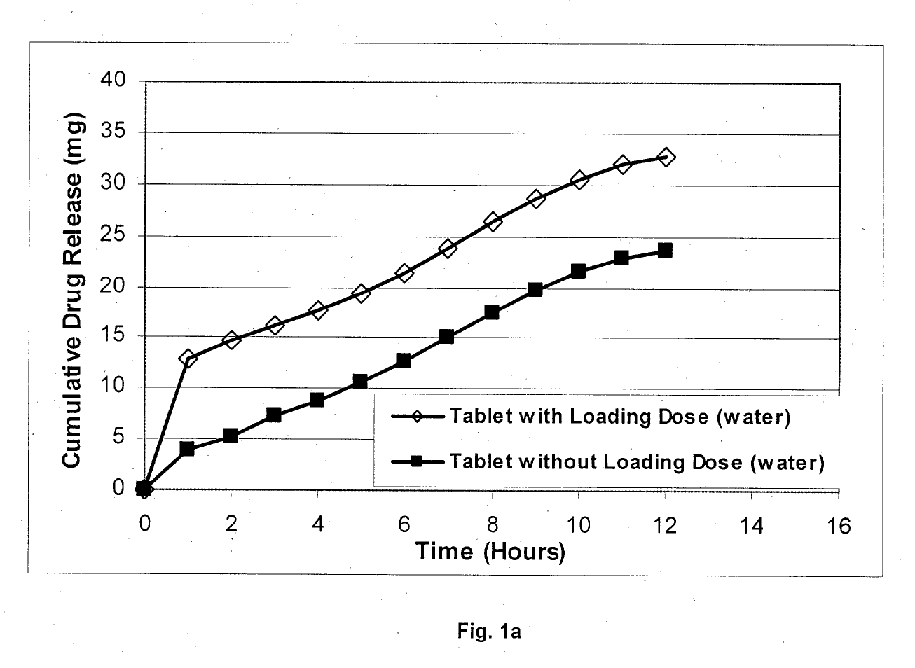

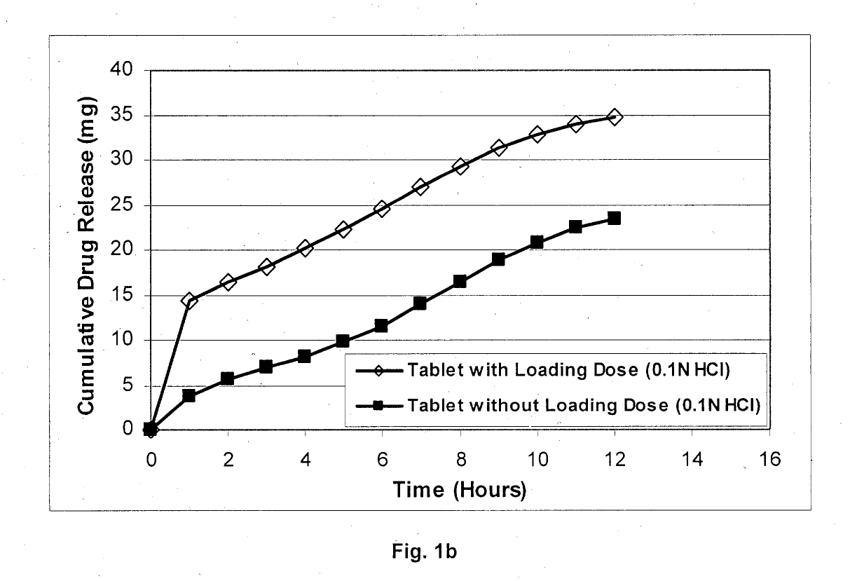

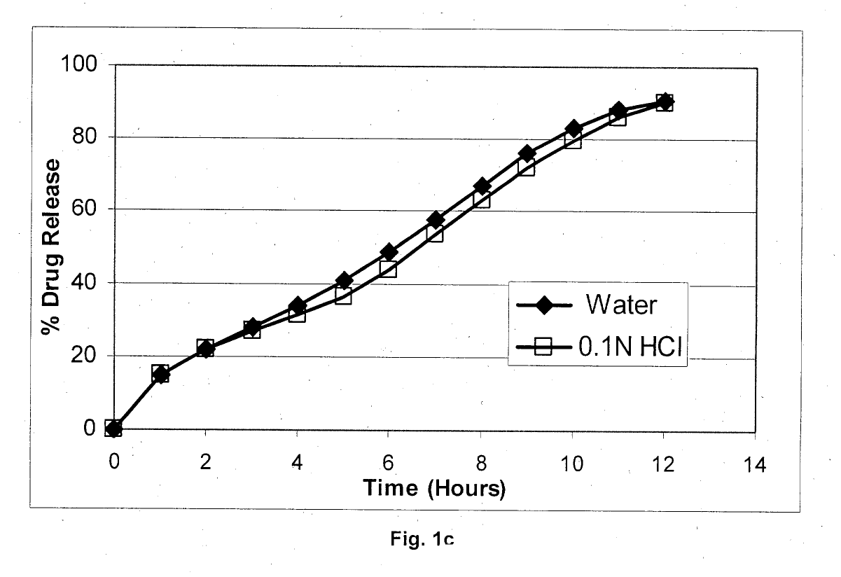

Active agent release rate data was obtained for these tablets using USP Apparatus 2 at 50 rpm using 900 ml of either water or 0.1 N HCl as the dissolution media. The results are shown in FIGS. 1(a) through 1(c).

### Example 2

The cores of Example 1 were used with a press-coat composition containing (per tablet):

| | 15% HPMC | 20% HPMC | 40% HPMC |
|---|---|---|---|
| Component | mg per tablet | mg per tablet | mg per tablet |
| Methylphenidate HCl | 10 | 10 | 10 |
| Lactose Monohydrate | 258 | 242.0 | 178 |
| METHOCEL K4M | 48 | 64.0 | 128 |
| Cab-O-Sil (silica) | 1 | 1.0 | 1 |
| Magnesium Stearate | 3 | 3.0 | 3 |
| Total Press-coat | 320 | 320.0 | 320 |

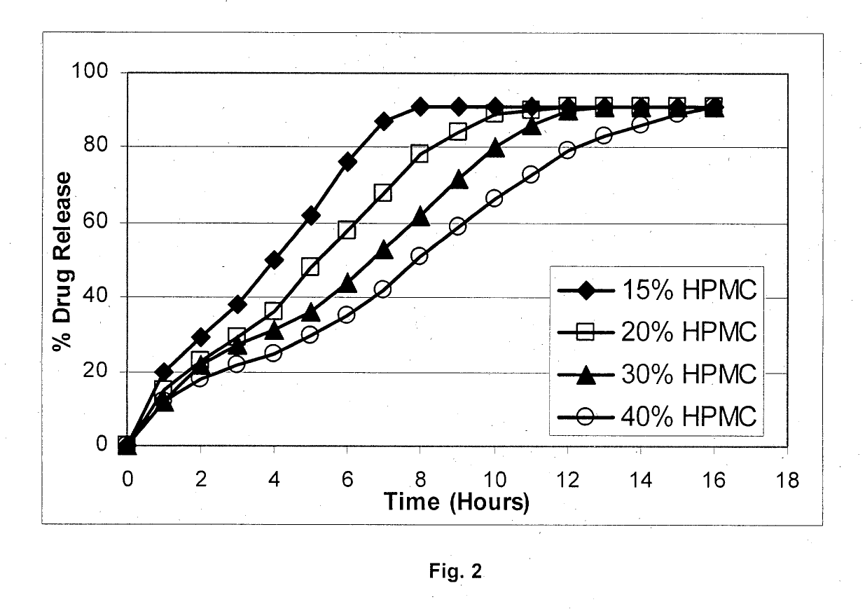

Tablets were prepared as in Example 1 (except that no loading dose was added) and dissolution profiles were measured as in Example 1. The results are set forth in FIG. 2, which also includes the corresponding information from the 30% HPMC composition of Example 1.

### Example 3

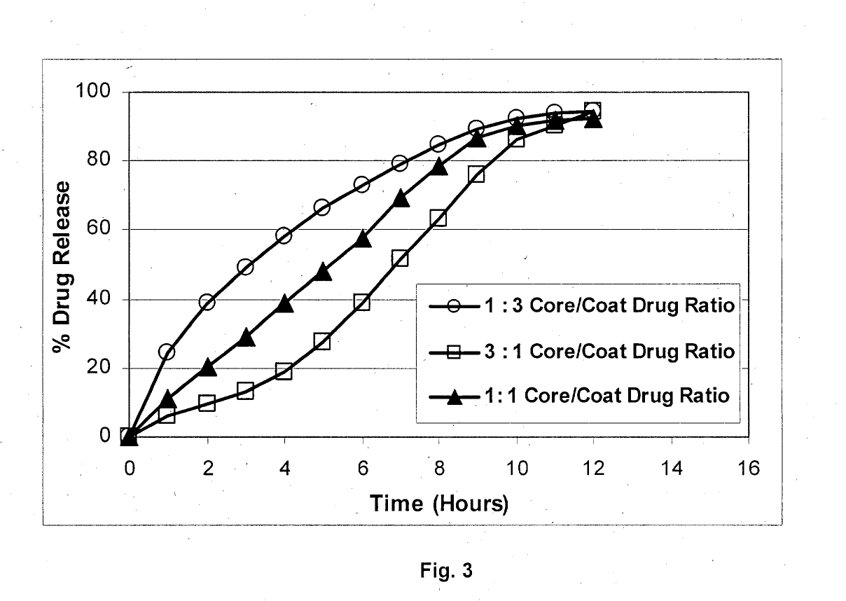

The cores of Example 1 were used with a press-coat composition comprising 12 mg of methylphenidate to provide a Core_{AA}:Coat_{AA} ratio of 4:3 (Example 3A). Additional methylphenidate hydrochloride formulations were prepared as set forth below to provide Core_{AA}:Coat_{AA} ratio of 1:3 (Example 3B) and 3:1 (Example 3C). The results of the drug dissolution testing in 0.1 N HCl are shown in FIG. 3.

| | mg per tablet | | |
|---|---|---|---|
| | Example 3A | Example 3B | Example 3C |
| Component | (4:3) | (1:3) | (3:1) |
| Core | | | |
| Methylphenidate HCl | 16 | 7.0 | 21.0 |
| Dicalcium Phosphate | 39.4 | 48.4 | 34.4 |
| Carnauba Wax | 24.0 | 24.0 | 24.0 |
| Cab-O-Sil (silica) | 0.2 | 0.2 | 0.2 |
| Magnesium Stearate | 0.4 | 0.4 | 0.4 |
| Total Core | 80.0 | 80.0 | 80.0 |
| Press-coating | | | |
| Methylphenidate HCl | 12 | 21.0 | 7.0 |
| Lactose Monohydrate | 248.0 | 239.0 | 253.0 |
| METHOCEL K4M | 56.0 | 56.0 | 56.0 |
| Cab-O-Sil (silica) | 1.0 | 1.0 | 1.0 |
| Magnesium Stearate | 3.0 | 3.0 | 3.0 |
| Total Press-coat | 320.0 | 320.0 | 320.0 |

### Example 4

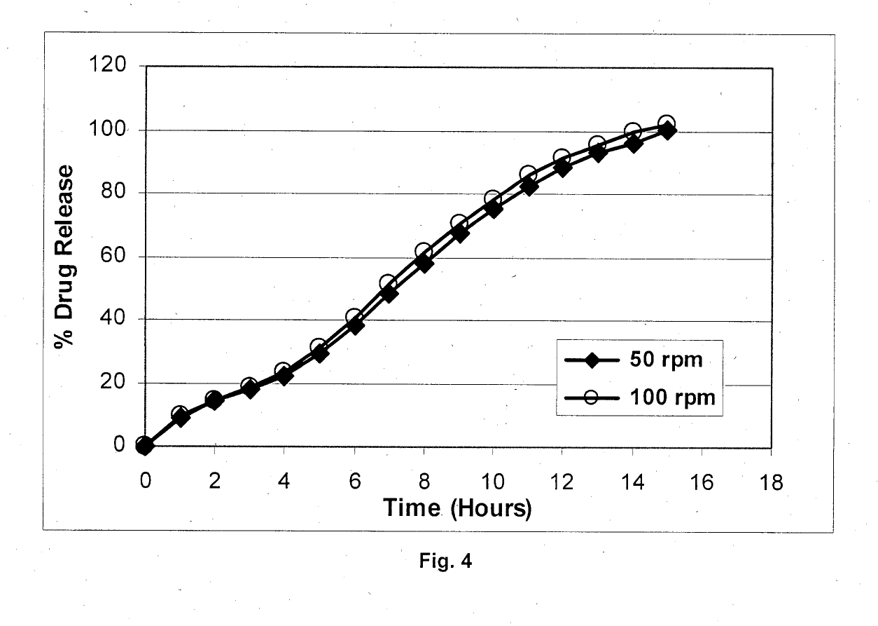

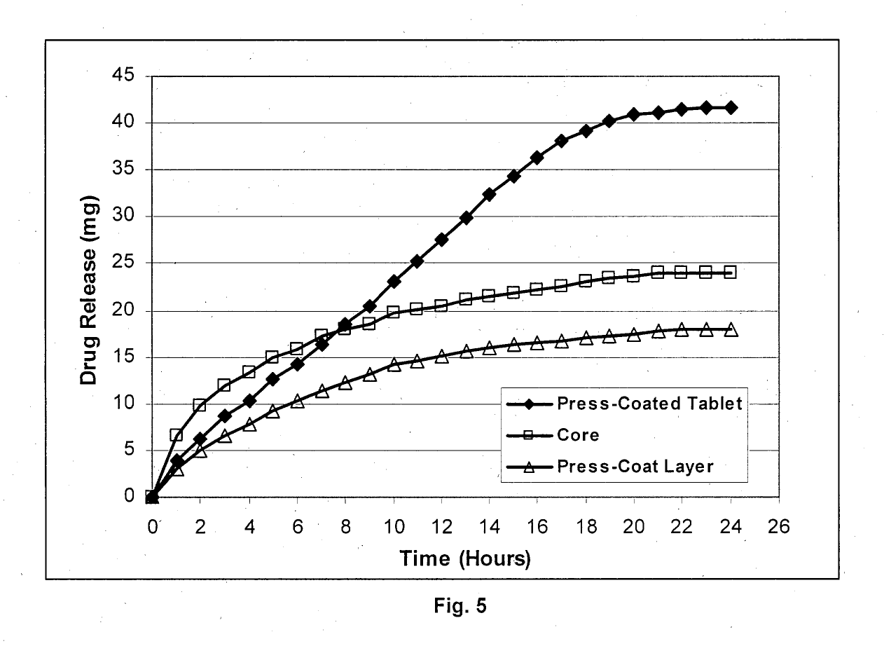

A methylphenidate hydrochloride formulation was prepared utilizing Methocel K100M mannitol and a Core_{AA}:Coat_{AA} ratio of 7:2. Dissolution data was obtained using two rotation speeds. The results are shown in FIG. 4.

| Component | mg per tablet |
|---|---|
| Methylphenidate HCl | 42.0 |
| Dicalcium Phosphate | 13.5 |
| Carnauba Wax | 14.0 |
| Magnesium Stearate | 0.5 |
| Total Core | 70.0 |
| Methylphenidate HCl | 12.0 |
| Mannitol granules (Pearlitol SD 200) | 231.5 |
| METHOCEL K100M | 82.5 |
| Cab-O-Sil (silica) | 1.0 |
| Magnesium Stearate | 3.0 |
| Total Press-coat | 330.0 |

### Example 5

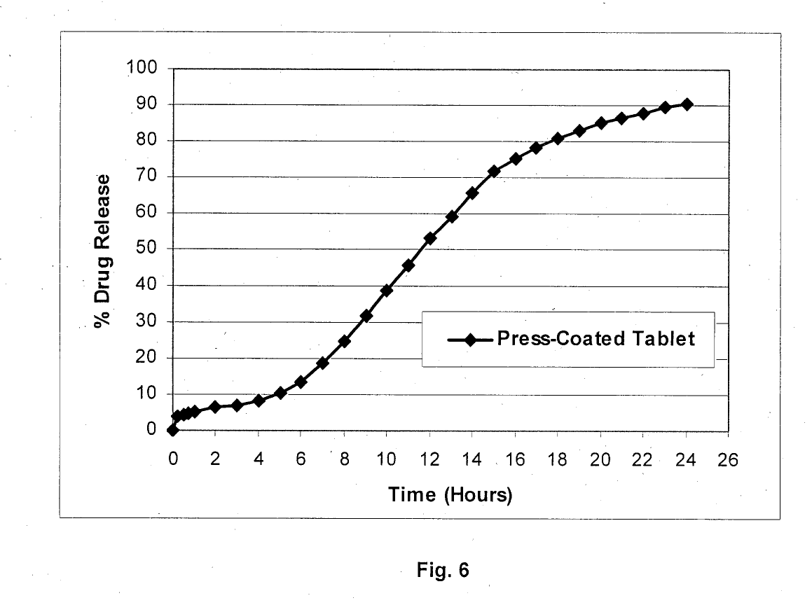

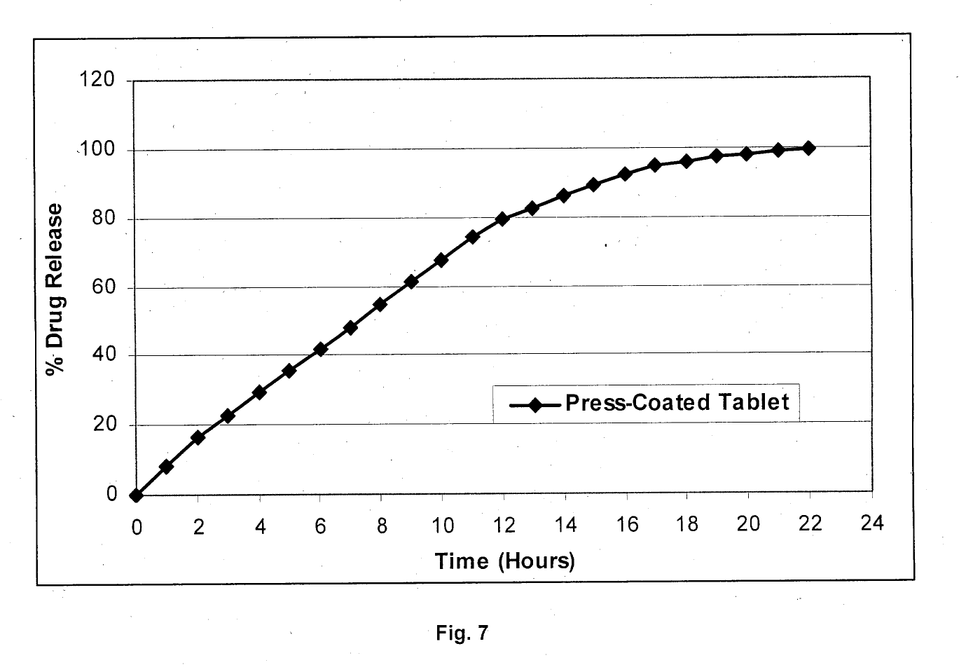

A fluoxetine hydrochloride formulation (Example 5A) and oxybutynin hydrochloride (Example 5B and 5C) formulations were also prepared. Dissolution data is shown in FIGS. 6-7.

| | mg per tablet | | |
|---|---|---|---|
| Component | Ex. 5A | Ex. 5B | Ex. 5C |
| Core | | | |
| Active Ingredient | 24.0 | 13.5 | 9.0 |
| Dicalcium Phosphate | 60.0 | | 25.0 |
| Carnauba Wax | 36.0 | 15.0 | 15.0 |
| Cab-O-Sil (silica) | | | |
| Talc | | 20.5 | |
| Magnesium Stearate | 0 | 1.0 | 1.0 |
| Total Core | 120.0 | 50.0 | 50.0 |
| Press-coating | | | |
| Active Ingredient | 18.0 | 0.75 | 6.0 |
| Lactose Monohydrate | 242.0 | | |
| Mannitol granules | | 100.00 | 165.5 |
| Dicalcium Phosphate | | 70.75 | |
| Anhyd. | | | |
| METHOCEL K4M | 114.0 | | |
| METHOCEL K100M | | 75.00 | 75.0 |
| Cab-O-Sil (silica) | 2.0 | 0.50 | 0.5 |
| Magnesium Stearate | 4.0 | 3.00 | 3.0 |
| Total Press-coat | 380.0 | 250.00 | 250.0 |
| Core_{AA}:Coat_{AA} ratio | 4:3 | 18:1 | 3:2 |
| % HPMC of Press-coat | 30% | 30% | 30% |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A press-coat composition comprising (a) a core composition comprising a water-soluble active agent and a waxy material and (b) a coating composition comprising the water-soluble active agent and a hydrophilic polymer, wherein the coating composition is press-coated onto the core composition.

2. The composition of claim 1, wherein the waxy material is carnauba wax, tribehenin, fatty alcohols, lauryl alcohol, myristyl alcohol, stearyl alcohol, palmityl alcohol, fatty acids, lauric acid, myristic acid, stearic acid, palmitic acid, polyethylenes, castor wax, C₁₆₋₃₀ fatty acid triglycerides, beeswax, or mixtures thereof.

3. The composition of claim 1, wherein the hydrophilic polymer comprises a hydrophilic cellulose polymer.

4. The composition of claim 1, wherein the hydrophilic polymer comprises a lower alkyl hydroxyalkyl cellulose polymer.

5. The composition of claim 1, wherein the hydrophilic polymer is selected from the group consisting of hydroxyethylcellulose (HEC), hydroxypropyl cellulose (HPC), hydroxypropylmethylcellulose (HPMC), hydroxypropylethylcellulose (HPEC), hydroxypropylpropylcellulose (HPPC), hydroxypropylbutylcellulose (HPBC), and mixtures thereof.

6. The composition of claim 1, wherein the active agent is selected from the group consisting of analgesics, anesthetics, anticonvulsants, antidiabetics, antihistamines, anti-infectives, antineoplastics, antiparkinsonian agents, antirheumatics, appetite stimulants, appetite suppressants, blood modifiers, bone metabolism modifiers, cardiovascular agents, central nervous system depressants, central nervous system stimulants, decongestants, dopamine receptor agonists, electrolytes, gastrointestinal agents, immunomodulators, muscle relaxants, narcotics, parasympathomimetics, sympathomimetics, sedatives, and hypnotics.

7. The composition of claim 1, wherein the active agent comprises a pharmaceutically acceptable salt of methylphenidate, fluoxetine, oxybutynin, or combinations thereof.

8. The composition of claim 7, wherein the active agent is a pharmaceutically acceptable salt of methylphenidate.

9. The composition of claim 7, wherein the active agent is a pharmaceutically acceptable salt of fluoxetine.

10. The composition of claim 7, wherein the active agent is a pharmaceutically acceptable salt of oxybutynin.

11. The composition of claim 1, wherein the active agent comprises methylphenidate hydrochloride, fluoxetine hydrochloride, oxybutynin chloride, or combinations thereof.

12. The composition of claim 1, wherein the ratio of the active agent in the core composition to the active agent in the coating composition is about 1:99 to about 99:1.

13. The composition of claim 1, wherein the ratio of the active agent in the core composition to the active agent in the coating composition is about 5:95 to about 95:5.

14. The composition of claim 1, wherein the ratio of the active agent in the core composition to the active agent in the coating composition is about 1:9 to about 9:1.

15. The composition of claim 7, wherein the ratio of the active agent in the core composition to the active agent in the coating composition is about 3:4 to about 5:3.

16. The composition of claim 7, wherein the ratio of the active agent in the core composition to the active agent in the coating composition less than about 3:4.

17. The composition of claim 7, wherein the ratio of the active agent in the core composition to the active agent in the coating composition is greater than about 5:3.

18. The composition of claim 1, wherein the active agent in the core composition and the active agent in the coating composition are present in an amount to provide a substantially zero order release profile.

19. The composition of claim 1, wherein the active agent in the core composition and the active agent in the coating composition are present in an amount to provide a substantially first order release profile.

20. The composition of claim 1, wherein the active agent in the core composition and the active agent in the coating composition are present in an amount to provide a substantially second order release profile.

21. The composition of claim 1, wherein the active agent in the core composition and the active agent in the coating composition are present in an amount wherein about 20% to about 50% of the active agent is dissolved within about 4 hours.

22. The composition of claim 1, wherein the active agent in the core composition and the active agent in the coating composition are present in an amount wherein about 25% to about 50% of the active agent is dissolved within about 4 hours.

23. The composition of claim 1, wherein the active agent in the core composition and the active agent in the coating composition are present in an amount wherein at least about 70% of the active agent is dissolved within about 12 hours.

24. The composition of claim 1, wherein the active agent in the core composition and the active agent in the coating composition are present in an amount wherein at least about 80% of the active agent is dissolved within about 12 hours.

25. The composition of claim 1, wherein the active agent in the core composition and the active agent in the coating composition are present in an amount wherein about 20% to about 50% of the active agent is dissolved within about 4 hours and at least about 70% of the active agent is dissolved within about 12 hours.

26. The composition of claim 1, wherein the active agent in the core composition and the active agent in the coating composition are present in an amount wherein about 25% to about 50% of the active agent is dissolved within about 4 hours and at least about 80% of the active agent is dissolved within about 12 hours.

27. The composition of claim 15, wherein the active agent is a pharmaceutically acceptable salt of methylphenidate or fluoxetine, and wherein the active agent in the core composition and the active agent in the coating composition are present in an amount wherein about 25% to about 50% of the active agent is dissolved within about 4 hours, and at least about 80% of the active agent is dissolved within about 12 hours.

28. The composition of claim 15, wherein the active agent is a pharmaceutically acceptable salt of oxybutynin, and wherein the active agent in the core composition and the active agent in the coating composition are present in an amount wherein about 20% to about 40% of the active agent is dissolved within about 4 hours, and at least about 70% of the active agent is dissolved within about 12 hours.

29. The composition of claim 16, wherein the active agent in the core composition and the active agent in the coating composition are present in an amount wherein about 40% to about 70% of the active agent is dissolved within about 4 hours, and at least about 85% of the active agent is dissolved within about 12 hours.

30. The composition of claim 17, wherein the active agent in the core composition and the active agent in the coating composition are present in an amount wherein about 5% to about 30% of the active agent is dissolved within about 4 hours, and at least about 90% of the active agent is dissolved within about 12 hours.

31. The composition of claim 1, wherein the active agent in the core composition and the active agent in the coating composition are present in an amount to provide a Tmax of about 3.5 to about 12 hours.

32. The composition of claim 1, wherein the active agent in the core composition and the active agent in the coating composition are present in an amount to provide a Tmax of about 4.5 to about 10 hours.

33. The composition of claim 1, wherein the active agent in the core composition and the active agent in the coating composition are present in an amount to provide a Tmax of about 6 to about hours.

34. The composition of claim 1, wherein the active agent in the core composition and the active agent in the coating composition are present in an amount to provide a Tmax of about 6.5 to about 7 hours.

35. The composition of claim 1, wherein the active agent in the core composition and the active agent in the coating composition are present in an amount to provide a Cmax of about 2.7 to about 4.9 ng/ml.

36. The composition of claim 35, wherein the active agent in the core composition and the active agent in the coating composition are present in an amount to provide a Cmax of about 3.2 to about 4.2 ng/ml.

37. The composition of claim 35, wherein the active agent in the core composition and the active agent in the coating composition are present in an amount to provide a Cmax of about 3.4 to about 3.9 ng/ml.

38. The composition of claim 35, wherein the active agent in the core composition and the active agent in the coating composition are present in an amount to provide a Cmax of about 3.6 to about 3.8 ng/ml.

39. The composition of claim 1, wherein the active agent in the core composition and the active agent in the coating composition are present in an amount to provide a Cmax of about 5 to about 10 ng/ml.

40. The composition of claim 39, wherein the active agent in the core composition and the active agent in the coating composition are present in an amount to provide a Cmax of about 5.5 to about 8 ng/ml.

41. The composition of claim 39, wherein the active agent in the core composition and the active agent in the coating composition are present in an amount to provide a Cmax of about 5.5 to about 7.5 ng/ml.

42. The composition of claim 39, wherein the active agent in the core composition and the active agent in the coating composition are present in an amount to provide a Cmax of about 6 to about 6.5 ng/ml.

43. The composition of claim 1, wherein the active agent in the core composition and the active agent in the coating composition are present in an amount to provide a Cmax of about 8 to about 15 ng/ml.

44. The composition of claim 43, wherein the active agent in the core composition and the active agent in the coating composition are present in an amount to provide a Cmax of about 8.5 to about 13 ng/ml.

45. The composition of claim 43, wherein the active agent in the core composition and the active agent in the coating composition are present in an amount to provide a Cmax of about 9 to about 12 ng/ml.

46. The composition of claim 1, wherein the active agent in the core composition and the active agent in the coating composition are present in an amount to provide an AUC of about 35 to about 45 (h)(ng/ml).

47. The composition of claim 46, wherein the active agent in the core composition and the active agent in the coating composition are present in an amount to provide an AUC of about 27.5 to about 43 (h)(ng/ml).

48. The composition of claim 46, wherein the active agent in the core composition and the active agent in the coating composition are present in an amount to provide an AUC of about 41.6 to about 42 (h)(ng/ml).

49. The composition of claim 1, wherein the active agent in the core composition and the active agent in the coating composition are present in an amount to provide an AUC of about 66.7 to about 70.5 (h)(ng/ml).

50. The composition of claim 49, wherein the active agent in the core composition and the active agent in the coating composition are present in an amount to provide an AUC of about 67.2 to about 70 (h)(ng/ml).

51. The composition of claim 49, wherein the active agent in the core composition and the active agent in the coating composition are present in an amount to provide an AUC of about 67.6 (h)(ng/ml).

52. The composition of claim 1, wherein the active agent in the core composition and the active agent in the coating composition are present in a an amount to provide a four hour post administration blood level of about 1.75 to about 3.75 ng/ml.

53. The composition of claim 52, wherein the active agent in the core composition and the active agent in the coating composition are present in a an amount to provide a four hour post administration blood level of about 1.9 to about 2.5 ng/ml.

54. The composition of claim 52, wherein the active agent in the core composition and the active agent in the coating composition are present in a an amount to provide a four hour post administration blood level of about 2 to about 2.25 ng/ml.

55. The composition of claim 1, wherein the active agent in the core composition and the active agent in the coating composition are present in a an amount to provide a four hour post administration blood level of about 5.5 to about 10 ng/ml.

56. The composition of claim 55, wherein the active agent in the core composition and the active agent in the coating composition are present in a an amount to provide a four hour post administration blood level of about 6 to about 9 ng/ml.

57. The composition of claim 10, wherein the active the active agent in the core composition and the active agent in the coating composition are present in an amount to provide a release rate of the active agent of about 0.05 to about 0.85 mg/hr over a substantial portion of a 24 hour period.

58. The composition of claim 10, wherein the active the active agent in the core composition and the active agent in the coating composition are present in an amount to provide a substantially zero order release rate of the active agent over a substantial portion of a 24 hour period.

59. The composition of claim 10, wherein the active the active agent in the core composition and the active agent in the coating composition are present in an amount to provide a yield plasma concentration over a substantial portion of a 24 hour period of about 4.2 to about 6.7 ng/ml.

60. The composition of claim 10, wherein the active agent in the core composition and the active agent in the coating composition are present in an amount to provide a ratio of oxybutynin to its desethyl metabolite of at least 0.18:1 over a substantial portion of a 24 hour period.

61. A press-coat composition comprising (a) a core composition comprising an active agent and carnauba wax and (b) a coating composition comprising the active agent and hydroxypropylmethyl cellulose (HPMC), wherein the active agent is selected from the group consisting of a pharmaceutically acceptable salt of methylphenidate, fluoxetine, oxybutynin, and mixtures thereof, and wherein the coating composition is press-coated onto the core.

62. A tablet dosage form comprising the composition of claim 1.

63. The composition of claim 1, further comprising (c) an additional coating composition comprising the pharmaceutically acceptable salt of methylphenidate.

64. The composition of claim 63, wherein the additional coating composition is an immediate release coating composition.

65. A press-coat composition comprising (a) a core composition comprising a water-soluble active agent and a waxy material, (b) a coating composition comprising the water-soluble active agent and a hydrophilic polymer, wherein the coating composition is press-coated onto the core, and (c) an additional coating composition comprising the water-soluble active agent.

66. A press-coat composition comprising (a) a core composition comprising a pharmaceutically acceptable salt of methylphenidate and a waxy material and (b) a coating composition comprising the pharmaceutically acceptable salt of methylphenidate and a hydrophilic polymer, wherein the coating composition is press-coated onto the core.

67. The composition of claim 63, wherein the pharmaceutically acceptable salt of methylphenidate is present in a weight ratio in the core composition:coating composition:additional coating composition of about 3:2:1.

68. The composition of claim 66, further comprising (c) an additional coating composition comprising the pharmaceutically acceptable salt of methylphenidate.

69. A press-coat composition comprising (a) a core composition comprising methylphenidate HCI and carnauba wax, (b) a coating composition comprising methylphenidate HCl and hydroxypropylmethyl cellulose (HPMC), wherein the coating composition is press-coated onto the core.

70. A press-coat composition comprising (a) a core composition comprising methylphenidate HCl and carnauba wax, (b) a coating composition comprising methylphenidate HCl and hydroxypropylmethyl cellulose (HPMC), wherein the coating composition is press-coated onto the core, and (c) an additional coating composition comprising methylphenidate HCl.

71. A method for preparing a press-coat composition, the method comprising (a) providing a core composition comprising a water-soluble active agent and a waxy material, (b) providing a coating composition comprising the water-soluble active agent and a hydrophilic polymer, and (c) press-coating the coating composition onto the core composition to provide the press-coat composition.

72. The method of claim 69, the method further comprising (d) coating an additional coating composition onto the press-coating composition, wherein the additional coating comprises the active agent.
